(19) United States
(12) Patent Application Publication
(10) Pub. No.: US 2009/0171198 A1
(43) Pub. Date: Jul. 2, 2009

(54) Powdered marker

(76) Inventors: Michael L. Jones, San Clemente, CA (US); Paul Lubock, Laguna Niguel, CA (US); John Merritt, San Clemente, CA (US)

Correspondence Address: EDWARD J. LYNCH;Patent Attorney, One Embarcadero Center, Suite 562, San Francisco, CA, 94111, US

(21) Appl. No.: 12/317,339

(22) Filed: Dec. 19, 2008

Related U.S. Application Data
(63) Continuation-in-part of application No. 11/881,264, filed on Jul. 26, 2007.
(60) Provisional application No. 60/835,740, filed on Aug. 4, 2006.

(51) Int. Cl. A61B 6/00; A61K 49/00

(52) U.S. Cl. 600/432; 424/9.1

(57) ABSTRACT

The marker delivery system described has a delivery tube or cannula which has a bioabsorbable powdered mass and a radiographically detectable element within an inner lumen thereof. The powdered material is a starch such as USP (corn) starch or other suitable polysaccharide. The radiographically detectable element may be disposed within or coupled to a bioabsorbable pellet.

### RELATED APPLICATIONS

This application is a continuation-in-part to U.S. application Ser. No. 11/881,264, filed on Jul. 26, 2007, which is related to and claims priority from provisional application Ser. No. 60/835,740, filed on Aug. 4, 2006, both of which are incorporated herein by reference in their entirety.

### FIELD OF THE INVENTION

The invention is generally directed to remotely detectable, intracorporeal markers and devices and methods for the delivery of such markers to a desired location within a patient's body.

### BACKGROUND OF THE INVENTION

In diagnosing and treating certain medical conditions, it is often desirable to mark a suspicious body site for the subsequent taking of a biopsy specimen, for delivery of medicine, radiation, or other treatment, for the relocation of a site from which a biopsy specimen was taken, or at which some other procedure was performed. As is known, obtaining a tissue sample by biopsy and the subsequent examination are typically employed in the diagnosis of cancers and other malignant tumors, or to confirm that a suspected lesion or tumor is not malignant. The information obtained from these diagnostic tests and/or examinations is frequently used to devise a therapeutic plan for the appropriate surgical procedure or other course of treatment.

In many instances, the suspicious tissue to be sampled is located in a subcutaneous site, such as inside a human breast. To minimize surgical intrusion into a patient's body, it is often desirable to insert a small instrument, such as a biopsy needle, into the body for extracting the biopsy specimen while imaging the procedure using fluoroscopy, ultrasonic imaging, x-rays, magnetic resonance imaging (MRI) or any other suitable form of imaging technique or palpation. Examination of tissue samples taken by biopsy is of particular significance in the diagnosis and treatment of breast cancer. In the ensuing discussion, the biopsy and treatment site described will generally be the human breast, although the invention is suitable for marking sites in other parts of the human and other mammalian body as well.

Periodic physical examination of the breasts and mammography are important for early detection of potentially cancerous lesions. In mammography, the breast is compressed between two plates while specialized x-ray images are taken. If an abnormal mass in the breast is found by physical examination or mammography, ultrasound may be used to determine whether the mass is a solid tumor or a fluid-filled cyst. Solid masses are usually subjected to some type of tissue biopsy to determine if the mass is cancerous.

If a solid mass or lesion is large enough to be palpable, a tissue specimen can be removed from the mass by a variety of techniques, including but not limited to open surgical biopsy, a technique known as Fine Needle Aspiration Biopsy (FNAB) and instruments characterized as “vacuum assisted large core biopsy devices”.

If a solid mass of the breast is small and non-palpable (e.g., the type typically discovered through mammography), a vacuum assisted large core biopsy procedure is usually used. In performing a stereotactic biopsy of a breast, the patient lies on a special biopsy table with her breast compressed between the plates of a mammography apparatus and two separate x-rays or digital video views are taken from two different points of view. A computer calculates the exact position of the lesion as well as depth of the lesion within the breast. Thereafter, a mechanical stereotactic apparatus is programmed with the coordinates and depth information calculated by the computer, and such apparatus is used to precisely advance the biopsy needle into the small lesion. The stereotactic technique may be used to obtain histologic specimens. Usually at least five separate biopsy specimens are obtained from locations around the small lesion as well as one from the center of the lesion.

The available treatment options for cancerous lesions of the breast include various degrees of mastectomy or lumpectomy, radiation therapy, chemotherapy and combinations of these treatments. However, radiographically visible tissue features, originally observed in a mammogram, may be removed, altered or obscured by the biopsy procedure, and may heal or otherwise become altered following the biopsy. In order for the surgeon or radiation oncologist to direct surgical or radiation treatment to the precise location of the breast lesion several days or weeks after the biopsy procedure was performed, it is desirable that a biopsy site marker be placed in the patient's body to serve as a landmark for subsequent location of the lesion site. A biopsy site marker may be a permanent marker (e.g., a metal marker visible under x-ray examination), or a temporary marker (e.g., a bioresorbable marker detectable with ultrasound). While current radiographic type markers may persist at the biopsy site, an additional mammography generally must be performed at the time of follow up treatment or surgery in order to locate the site of the previous surgery or biopsy. In addition, once the site of the previous procedure is located using mammography, the site must usually be marked with a location wire which has a hook on the end which is advanced into site of the previous procedure. The hook is meant to fix the tip of the location wire with respect to the site of the previous procedure so that the patient can then be removed from the confinement of the mammography apparatus and the follow-up procedure performed. However, as the patient is removed from the mammography apparatus, or otherwise transported the position of the location wire can change or shift in relation to the site of the previous procedure. This, in turn, can result in follow-up treatments being misdirected to an undesired portion of the patient's tissue.

As an alternative or adjunct to radiographic imaging, ultrasonic imaging (herein abbreviated as “USI”) or visualization techniques can be used to image the tissue of interest at the site of interest during a surgical or biopsy procedure or follow-up procedure. USI is capable of providing precise location and imaging of suspicious tissue, surrounding tissue and biopsy instruments within the patient's body during a procedure. Such imaging facilitates accurate and controllable removal or sampling of the suspicious tissue so as to minimize trauma to surrounding healthy tissue.

For example, during a breast biopsy procedure, the biopsy device is often imaged with USI while the device is being inserted into the patient's breast and activated to remove a sample of suspicious breast tissue. As USI is often used to image tissue during follow-up treatment, it may be desirable to have a marker, similar to the radiographic markers discussed above, which can be placed in a patient's body at the site of a surgical procedure and which are visible using USI. Such a marker enables a follow-up procedure to be performed without the need for traditional radiographic mammography imaging which, as discussed above, can be subject to inaccuracies as a result of shifting of the location wire as well as being tedious and uncomfortable for the patient.

Placement of a marker or multiple markers at a location within a patient's body requires delivery devices capable of holding markers within the device until the device is properly situated within a breast or other body location. Accordingly, devices and methods for retaining markers within a marker delivery device while allowing their expulsion from the devices at desired intracorporeal locations are desired.

In addition to marking functions, frequently it is desirable to provide treatments with the marker members such as hemostatic treatment and the like.

### SUMMARY OF THE INVENTION

The invention is generally directed to a remotely imageable marker system suitable for deployment at a site within a patient's body, particularly a biopsy site such as in a patient's breast. The imageable marker system includes a mass of powdered starch or other polysaccharide sufficient to accelerate thrombus formation at the site where tissue has been removed. The starch or other suitable polysaccharide has a molecular weight of about 3500 to about 200,000 Daltons and is preferably a dry powder having a particle size of about 20-100 micrometers. The marker system also includes a radiopaque element preferably coupled to or disposed within a pellet formed of bioabsorbable material.

The powdered starch or other polysaccharide rapidly absorbs fluid and hydrates and in the process dehydrates blood at the site of deployment to rapidly initiate the clotting process. The radiopaque element provides long term identification of the intracorporeal site. Preferably, the radiopaque element is formed of non-magnetic material to avoid interference with magnetic resonance imaging (MRI). Suitable non-magnetic materials include titanium, platinum, gold, iridium, tantalum, tungsten, silver, rhodium, non-magnetic stainless steel (316) and the like. The radiopaque element should have a non-natural shape so that it is readily recognized when remotely imaged and should have a maximum dimension of about 0.5 to about 5 mm, preferably about 1 to about 3 mm to ensure remote identification, particularly with MRI.

The amount of powdered starch or other polysaccharide delivered to the site generally ranges from about 0.002 to about 0.01 in³ (0.0003-0.0016 cm³), preferably about 0.003 to about 0.008 in³ (0.0005-0.0013 cm³). The bioabsorbable pellet(s) to which radiopaque elements are coupled or disposed within will generally be about 0.2 to about 3 mm, preferably about 1 to about 2 mm, in diameter and about 3 to about 7 mm, preferably about 4 to about 6 mm in length. The pellets may be formed of bioabsorbable materials such as gelatin, polylactic acid, polyglycolic acid, polycaprolactone and copolymers thereof. Other suitable bioabsorbable materials may be used.

The marker member embodying features of the invention can be readily delivered to the desired location by suitable delivery systems such as disclosed in co-pending application Ser. No. 10/444,770, filed on May 23, 2003 and Ser. No. 10/753,277, filed on Dec. 23, 2003. The marker delivery system generally has an elongated cannula or syringe-like body with proximal and distal ports and an inner lumen extending between the ports. The powdered mass of the marker is slidably disposed within the inner lumen of the delivery cannula and a plunger slidably disposed within the inner lumen of the delivery cannula proximal to the markers. The plunger is movable from an initial position proximal to the markers within the tube, to a delivery position close to the discharge opening in the distal end of the cannula to push the marker members out of the discharge opening into the target tissue site. The radiopaque marker element or bioabsorbable pellet having such marker element is preferably disposed within the powdered mass but may be proximal or distal to the powdered mass.

Upon being discharged into the intracorporeal target site, the starch or other suitable polysaccharide quickly takes up body fluid at the site initiating the clotting process. The marker mass at least partially fills the site to enable short term detection (at least three weeks, preferably at least four weeks but less than a year) by remote USI and the radiopaque element provides long term detection by remote mammographic imaging or MRI identification.

The cannula of the marker delivery device may be configured to fit within the guide cannula of a biopsy device, such as the SenoCor 360% biopsy device sold by SenoRx (the present assignee), the EnCor™ biopsy device sold by SenoRx, a Mammotomee (sold by Johnson & Johnson), the ATEC biopsy device sold by Suros (Hologic) and or a coaxial needle guide. The delivery cannula can also be configured to fit into the proximal end of a tubular cutting element such as found in the EnCor™ biopsy system sold by SenoRx which is the subject of co-pending application Ser. No. 10/911,106, filed on Aug. 3, 2004.

One suitable delivery system suitable for delivery through a tubular cutter (e.g. as with the Encor™ system) is a syringe-type delivery system described in co-pending application Ser. No. 10/911,106, filed on Aug. 3, 2004 having a tubular shaft with a flared guide on or integral with the distal tip to facilitate engagement with the proximal end of the tubular cutter. Another syringe-type delivery system has a plugged or partially plugged distal tip to prevent body fluids from engaging one or more markers which may be in the tubular shaft of the delivery system. Such fluid infusions can retard or restrict discharging the powdered marker and other markers which may be within the inner lumen of the delivery cannula. Delivery systems with plugged or partially plugged tips are described in co-pending application Ser. No. 10/444,770, filed on May 23, 2003 and Ser. No. 10/753,277, filed on Dec. 23, 2003, which are incorporated herein in their entireties. The plugged tip type delivery systems can have a side opening for marker deployment or a plugged needle-type distal tip both of which are disclosed in the above application Ser. No. 10/753,694.

A variety of therapeutic or diagnostic agents may be incorporated into the powdered marker mass. Incorporated agents can include for example, additional hemostatic agents to form thrombus at the intracorporeal site, anesthetic agents to control pain, chemotherapeutic agents for treating residual neoplastic tissue or coloring agents to facilitate subsequent visual location of the site. Antibiotics, antifungal agents and antiviral agents may also be incorporated into the fibrous marker.

The radiopaque marker element or pellet coupled to or containing such an element is stabilized quickly in the intra-cavity clot which forms at the biopsy site and can be readily identified from surrounding tissue of the cavity.

These and other advantages of the invention will become more apparent from the following detailed description of embodiments when taken in conjunction with the accompanying exemplary drawings.

### BRIEF DESCRIPTION OF THE DRAWINGS

FIG. 1 is a perspective view, partially in longitudinal section, of a powdered marker delivery assembly embodying features of the invention.

FIG. 2 is a transverse cross-sectional view of the marker delivery assembly of FIG. 1 taken at line 2-2.

FIG. 3 is a transverse cross-sectional view of the marker delivery assembly of FIG. 1 taken at line 3-3.

FIG. 4 is a perspective view, partially in section, of a human breast from which a biopsy specimen has been removed, showing a powdered marker mass and radiographically detectable pellet delivered to the biopsy site.

FIG. 5 is a partial, longitudinal cross-section of the cannula shown in FIG. 1 with a plurality radiographically detectable pellets.

### DETAILED DESCRIPTION OF EMBODIMENTS OF THE INVENTION

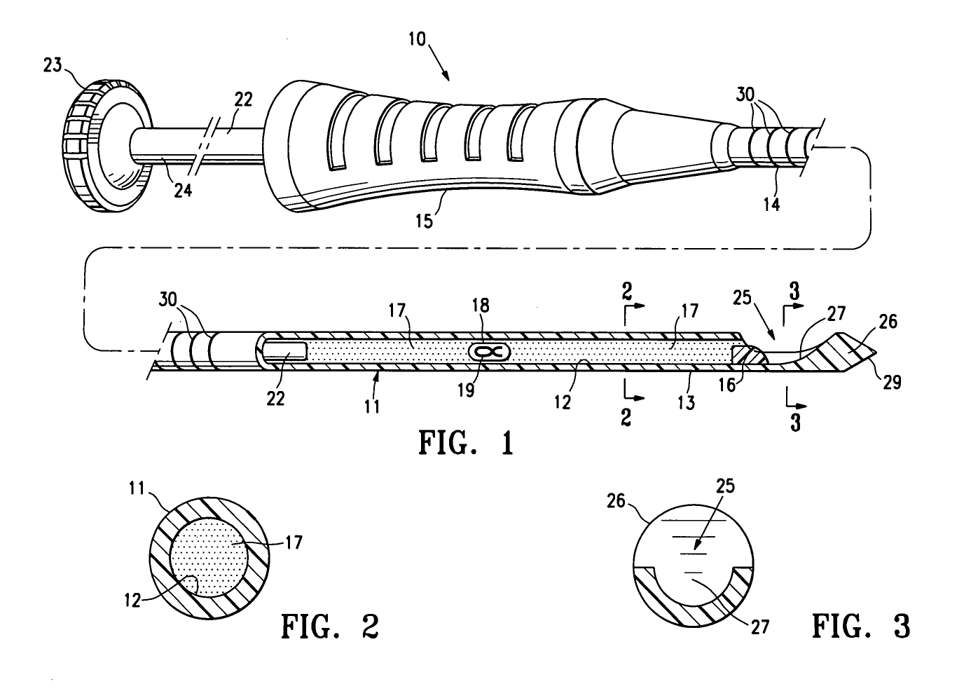

FIGS. 1-3 illustrate a powdered marker delivery system 10 embodying features of the invention which includes a delivery tube or cannula 11 with an inner lumen 12, a distal portion 13, and a proximal portion 14 with a handle 15. A releasable distal plug 16, powdered marker masses 17 and radiographically detectable pellet 18 with radiopaque element 19 are shown disposed within the inner lumen 12. A plunger 22 is slidably disposed within the inner lumen 12 and is provided with a head 23 on the proximal end 24 configured to allow an operator to press the plunger further into the inner lumen and push the releasable plug 16, powdered marker masses 17 and pellet 18 out of the discharge port or opening 25 in the distal end 26 of delivery cannula 11. Cannula handle 15 allows an operator to hold the cannula 11 steady while pressing plunger 22.

Releasable plug 16 may occupy or block only a portion of the discharge opening 25 as shown in FIG. 1 or it may completely fill or occlude the discharge opening. The exposed face of plug 16 is preferably provided with an inclined configuration. Releasable plug 16 is configured to be tight enough, e.g. press fit, in the inner lumen 12 to prevent its inadvertent release which might allow premature discharge of marker masses 17 from delivery cannula 11, but the plug must be easily released when the plunger 22 is pressed deeper into the inner lumen 12 of the delivery cannula 11. An adhesive or mechanical element(s) may be used to hold the releasable plug 16 in a position within the inner lumen 12. Suitable adhesives include polyurethane or polyacrylic based adhesives, polyhydroxymethacrylate base adhesives, fibrin glue (e.g., Tisseal™), collagen adhesive, or mixtures thereof. Suitable mechanical means for securing the releasable plug 16 are described in co-pending application Ser. No. 10/174,401. The distal end 26 of the delivery cannula 11 is provided with a ramp 27 which guides the discharged plug 16, powdered marker masses 17 and pellet 18 out of the side port 25 into the target site. The distal tip 29 may be tapered for delivery through a guide tube (not shown).

The delivery cannula 11 may be provided with markings 30 which serve as visual landmarks to aid an operator in accurately placing the distal portion 13 of the cannula 11 in a desired location within a patient's body for discharging the powdered marker masses 17 and radiographically detectable pellet 18.

The exterior of the delivery cannula 11 is preferably configured to fit within a guide cannula sized to accept a Mammotome®, Tru-Cut®, SenoCor®, EnCor™ or the ATEC biopsy device. Typically, plug 16 and pellet 18 will have diameters determined by the size of the inner lumen 12 and typically will be about 0.02 inch (0.5 mm) to about 0.5 inch (12 mm), preferably about 0.04 inch (1 mm) to about 0.3 inch (8 mm). Plug 16 may have slightly larger transverse dimensions to provide a tight fit.

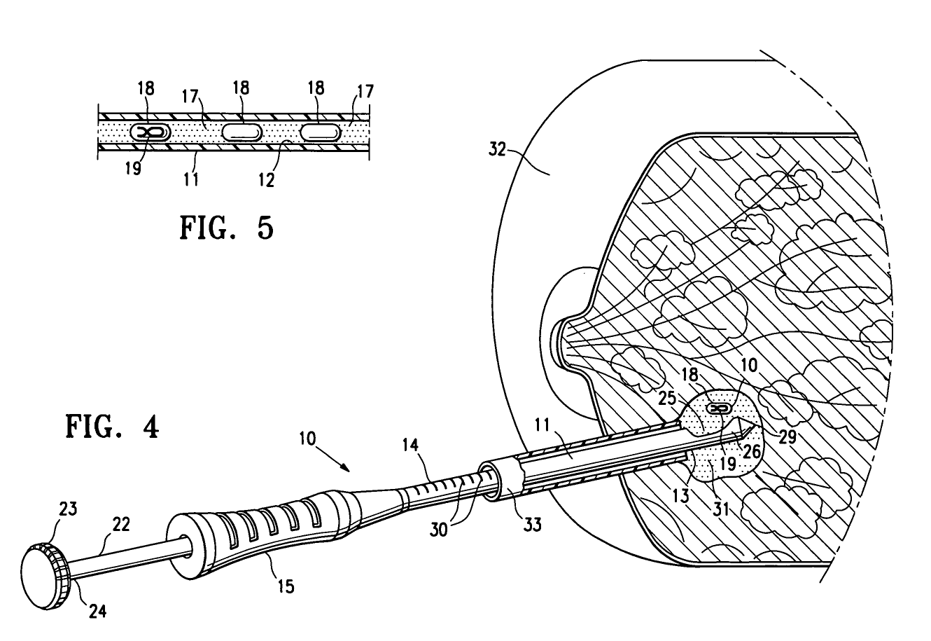

FIG. 4 schematically illustrates the delivery of powdered marker masses 17 and radiographically detectable pellet 18 to a cavity 31 such as a biopsy site in a patient's body. The distal portion of the cannula 11 of the marker delivery system 10 is shown inserted into a breast 32 through a guide cannula 33 until the distal end is disposed in the cavity 31 where a tissue specimen has been removed. While an operator holds the system 10 by the handle 15 of the delivery cannula 11, the plunger 22 is pressed further into the bore 12 of delivery cannula 11 to discharge the releasable plug 16, powdered marker masses 17 and pellet 18 into the cavity 31. FIG. 4 schematically illustrates the powdered marker mass 17 within the cavity 31 after deployment. When the powdered marker mass 17 contacts body fluid, such as blood within the cavity 31, moisture is drawn away from the blood or other fluid and the clotting cascade begins to form thrombus at the site. The bioabsorbable pellet 18 remains at the site and is remotely detectable for about three weeks to about 2 months but is gradually absorbed into the patient's body. The radiopaque element 19 within the pellet 18 remains at the site long term after the absorption of the pellet 18 to ensure subsequent relocation of the site 31.

Suitable starch/polysaccharide material include USP (corn) starch and Hemaderm™ which is available from Medafor, Inc. located in Minneapolis, Minn. These products and other suitable powdered products are described at least in part in U.S. Pat. No. 6,060,461, which is incorporated herein by reference. The amount of powdered starch or other suitable polysaccharide comprising the powdered marker mass(es) 17 is sufficient to substantially fill the biopsy site and to cause rapid clotting upon delivery thereto. Typically, the minimum amount of starch to water to form a suitable gel is at least 5%, preferably at least about 10% (wt %).

FIG. 5 illustrates a plurality of spaced apart pellets 18 within powdered masses 17 disposed in inner lumen 12. As shown, individual powder masses 17 may be proximal and/or distal to pellet 18.

While one or more particular forms of the invention have been illustrated and described herein in the context of a breast biopsy site, it will be apparent that the device and methods having features of the invention may find use in a variety of locations and in a variety of applications, in addition to the human breast, where tissue has been removed. Moreover, various modifications can be made without departing from the spirit and scope of the invention. Accordingly, it is not intended that the invention be limited to the specific embodiments illustrated. It is therefore intended that this invention to be defined by the scope of the appended claims as broadly as the prior art will permit, and in view of the specification if need be. Moreover, those skilled in the art will recognize that features shown in one embodiment may be utilized in other embodiments. Additional details of pellet or fibrous marker members and delivery systems may be found in co-pending application Ser. No. 10/753,694, filed on Jan. 7, 2004, and Ser. No. 10/976,138, filed on Oct. 27, 2004. Terms such as “element”, “member”, “device”, “section”, “portion”, “step”, “means” and words of similar import when used in the following claims shall not be construed as invoking the provisions of 35 U.S.C. §112(6) unless the following claims expressly use the term “means” followed by a particular function without specific structure or expressly use the term “step” followed by a particular function without specific action. All patents and patent applications referred to above are hereby incorporated by reference in their entirety.

What is claimed is:

1. A biopsy site marker delivery system, comprising:
a. an elongated tubular shaft which has a distal end, a proximal end, an inner lumen extending between the proximal and distal ends and a discharge opening in a distal shaft section;
b. at least one remotely detectable mass of powdered bioabsorbable material slidably disposed in the inner lumen of the shaft and comprising at least in part a starch or a suitable polysaccharide; and
c. a radiographically detectable marker element slidably disposed in the inner lumen of the shaft; and
d. a plunger element which is slidably disposed in part within the inner lumen of the tubular shaft which is configured to urge the mass of powdered bioabsorbable material and the radiographically detectable marker element out the discharge opening in the distal shaft section of the elongated tubular shaft.

2. The biopsy site marker delivery system of claim 1 wherein the radiographically detectable marker element is coupled to or disposed within a pellet formed of bioabsorbable material.

3. The biopsy site marker delivery system of claim 2 wherein the powdered bioabsorbable material has a faster absorption rate than the bioabsorbable material of the pellet.

4. The biopsy site marker delivery system of claim 1 wherein at least one remotely detectable mass of the powdered bioabsorbable material is disposed distal to the radiographically detectable marker element.

5. The biopsy site marker delivery system of claim 1 wherein at least one remotely detectable mass of the powdered bioabsorbable material is disposed proximal to the radiographically detectable marker element.

6. The biopsy site marker delivery system of claim 1 wherein at least one remotely detectable mass of the powdered bioabsorbable material is disposed distal to the radiographically detectable marker element and at least one remotely detectable mass of the powdered bioabsorbable material is disposed proximal to the radiographically detectable marker element.

7. The biopsy site marker delivery system of claim 1 wherein the plunger element is proximal to the mass of powdered bioabsorbable material and the radiographically detectable marker element.

8. The biopsy site marker system of claim 1 wherein the bioabsorbable starch or other suitable polysaccharide exhibits hemostatic properties.

9. The biopsy site marker system of claim 1 wherein the radiopaque element is non-magnetic.

10. The biopsy site marker system of claim 9 wherein the radiopaque element is formed of a metal selected from the group consisting of titanium, platinum, gold, iridium, tantalum, tungsten, silver, rhodium and non-magnetic stainless steel.

11. The biopsy site marker system of claim 1 wherein the powdered mass has a molecular weight of about 3500 to about 200,000 Daltons.

12. The biopsy site marker system of claim 1 wherein the powdered mass has a particle size of about 20-100 micrometers.

13. The biopsy site marker system of claim 1 wherein the pellet is formed of bioabsorbable material selected from the group consisting of gelatin, polylactic acid, polyglycolic acid, copolymers of polylactic acid and polyglycolic acid synthetic polymeric material comprises polyglycolic acid.

14. The biopsy site marker system of claim 1 wherein the volume of the powdered mass within the inner lumen of the cannula is 0.002 to about 0.01 in³ (0.0003-0.0016 cm³),

15. The biopsy site marker system of claim 1 wherein the volume of the powdered mass within the inner lumen of the cannula is about 0.003 to about 0.008 in³ (0.0005-0.0013 cm³).

16. A biopsy site marker delivery system, comprising:
a. an elongated tubular shaft which has a distal end, a proximal end, an inner lumen extending between the proximal and distal ends and a discharge opening in a distal shaft section;
b. at least one site marker member slidably disposed in the inner lumen of the shaft, comprising at least in part starch or a suitable polysaccharide; and
c. a plunger element which is slidably disposed in part within the inner lumen of the tubular shaft proximal to the site marker and which is configured to urge the site marker out the discharge opening in the distal shaft section of the elongated tubular shaft.

17. The biopsy site marker delivery system of claim 16 wherein the distal end of the elongated shaft is blocked by a releasable plug.

18. The biopsy site marker delivery system of claim 17 wherein the marker member has a radiopaque element.

19. The biopsy site marker delivery system of claim 18 wherein the radiopaque element is secured to a central portion of the marker member.

20. The biopsy site marker delivery system of claim 19 wherein the radiopaque element at least in part encircles a central portion of the marker member.

21. The biopsy site marker delivery system of claim 20 wherein the marker member containing starch or a suitable polysaccharide is a fibrous marker member.

22. The biopsy site marker delivery system of claim 21 wherein the marker member containing starch or a suitable polysaccharide has a pellet shape.

23. A method for delivering at least one marker member to an intracorporeal site within a patient from which tissue has been removed or separated from surrounding tissue, comprising:
a. providing a marker delivery device for delivery of at least one marker member which includes;
i. an elongated shaft which has an inner lumen, a discharge opening in a distal portion of the elongated shaft;
ii. at least one marker member which is formed at least in part of starch or a suitable polysaccharide and which is slidably disposed within the inner lumen of the elongated shaft; and
iii. a plunger element which is slidably disposed within the inner lumen of the marker delivery device proximal to the at least one marker member disposed therein and which is configured to urge the at least one marker member out the discharge opening in the distal portion of the elongated shaft;
b. advancing the marker delivery device within the patient until the distal end of the marker delivery device is disposed at the target tissue site and the discharge opening of the marker delivery device is aligned for marker member deployment; and
c. pressing the plunger element of the marker delivery device to eject the fibrous marker body through the discharge opening in the marker delivery device and into the target site.
